(19) United States
(12) Patent Application Publication
(10) Pub. No.: US 2025/0268882 A1
(43) Pub. Date: Aug. 28, 2025

(54) NEUROPATHIC AND CHRONIC PAIN FORMULATIONS

(71) Applicant: AAPEX Community Pharmacy, Inc., Houston, TX (US)

(72) Inventors: Jaimson ABRAHAM, Sugar Land, TX (US); Edward Joseph Lis, Katy, TX (US)

(73) Assignee: AAPEX Community Pharmacy, Inc., Houston, TX (US)

(21) Appl. No.: 19/056,992

(22) Filed: Feb. 19, 2025

Related U.S. Application Data
(60) Provisional application No. 63/557,189, filed on Feb. 23, 2024.

(51) Int. Cl. A61K 31/445; A61K 31/135; A61K 31/167; A61K 31/192; A61K 31/196; A61K 31/197; A61K 31/4168

(52) U.S. Cl. CPC A61K 31/445; A61K 31/135; A61K 31/167; A61K 31/192; A61K 31/196; A61K 31/197; A61K 31/4168

(57) ABSTRACT

A composition for treatment of pain and inflammation in humans includes 4.0-10.0% Diclofenac Sodium by weight, 2.0-6.0% Gabapentin by weight, 1.0-2.0% Baclofen by weight, and 2.0-5.0% Cyclobenzaprine by weight. Other compositions are also disclosed.

### CROSS-REFERENCE TO RELATED APPLICATIONS

This patent application claims the benefit and incorporates by reference the entire disclosure of U.S. Patent Application No. 63/557,189.

### TECHNICAL FIELD

The present disclosure relates generally to pharmaceutical compositions and more particularly, but not by way of limitation, to pharmaceutical compositions for use in treatment of neuropathic pain and inflammation in humans.

### BACKGROUND

This section provides background information to facilitate a better understanding of the various aspects of the disclosure. It should be understood that the statements in this section of this document are to be read in this light and not as admissions of prior art.

Many individuals suffer from neuropathic pain and inflammation due to a variety of conditions. There is a need for improved treatment and management of neuropathic pain and inflammation in such individuals.

### SUMMARY

This summary is provided to introduce a selection of concepts that are further described below in the detailed description. This summary is not necessarily intended to identify key or essential features of the claimed subject matter, nor is it intended to be used as an aid in limiting the scope of claimed subject matter.

A composition for treatment of pain and inflammation in humans includes 4.0-10.0% Diclofenac Sodium by weight, 2.0-6.0% Gabapentin by weight, 1.0-2.0% Baclofen by weight, and 2.0-5.0% Cyclobenzaprine by weight.

A composition for treatment of pain and inflammation in humans includes 0.5-1.0% Bupivacaine by weight, 2.0-6.0% Gabapentin by weight, 1.0-2.0% Baclofen by weight, 1.0-2.0% Cyclobenzaprine by weight, and 3.0-5.0% Diclofenac Sodium by weight.

A composition for treatment of pain and inflammation in humans includes 5.0-10.0% Ketoprofen by weight, 1.0-2.0% Baclofen by weight, 1.0-5.0% Gabapentin by weight, and 4.0-6.0% Lidocaine by weight.

A composition for treatment of pain and inflammation in humans includes 5.0-10.0% Flurbiprofen by weight, 1.0-5.0% Cyclobenzaprine by weight, 2.0-6.0% Gabapentin by weight, 1.0-2.0% Lidocaine by weight, and 1.0-2.0% Prilocaine by weight.

A composition for treatment of pain and inflammation in humans includes 2.0-6.0% Gabapentin by weight, 1.0-2.5% Lidocaine by weight, and 0.1-0.2% Clonidine by weight.

### DETAILED DESCRIPTION

Various embodiments will now be described more fully. The disclosure may, however, be embodied in many different forms and should not be construed as limited to the embodiments set forth herein.

A first group of formulations relates to different pharmaceutical compositions for use in the treatment of neuropathic pain and inflammation in humans. Various compositions include an effective amount of nerve blockers, muscle relaxants, and analgesics. The percentages below are by weight.

Illustrative formulations include the following:
NCP-2 Cream—Diclofenac Sodium 4.0%- 10.0%, Gabapentin 2.0%-6.0%, Baclofen 1.0%- 2.0%, and Cyclobenzaprine 2.0%- 5.0%
NCP-5 Cream—Bupivacaine 0.5%- 1.0%, Gabapentin 2.0%- 6.0%, Baclofen 1.0%- 2.0%, Cyclobenzaprine 1.0%- 2.0%, and Diclofenac Sodium 3.0%- 5.0%

A second group of formulations relates to different pharmaceutical compositions for use in the treatment of pain and inflammation in humans. Various compositions include an effective amount of nerve blockers, muscle relaxants, and analgesics.

Illustrative formulations include the following:
GPI-2 Cream—Ketoprofen 5.0%- 10.0%, Baclofen 1.0%- 2.0%, Gabapentin 1.0%- 5.0%, and Lidocaine 4%-6%
GPI-3 Cream—Flurbiprofen 5.0%- 10.0%, Cyclobenzaprine 1.0%- 5.0%, Gabapentin 2.0%- 6.0%, Lidocaine 1.0%- 2.0%, and Prilocaine 1.0%- 2.0%

A third group of formulations relates to different pharmaceutical compositions for use in the treatment of pain in the back due, for example, to spinal nerve compression or inflammation in humans. Various compositions include an effective amount of nerve blockers, muscle relaxants, and an analgesic.

An illustrative formulation is as follows:
BRP-1 Cream—Gabapentin 2.0%- 6.0%, Lidocaine 1.0%- 2.5%, and Clonidine 0.1%- 0.2%

In a typical embodiment, the compositions are in the form of a cream or lotion. Illustrative active ingredients comprise those listed above. Various additional ingredients may include, for example, inactive ingredients such as a base to incorporate all the active ingredients.

Depending on the embodiment, certain acts, events, or functions of any of the algorithms described herein can be performed in a different sequence, can be added, merged, or left out altogether (e.g., not all described acts or events are necessary for the practice of the algorithms). Moreover, in certain embodiments, acts or events can be performed concurrently, e.g., through multi-threaded processing, interrupt processing, or multiple processors or processor cores or on other parallel architectures, rather than sequentially. Although certain computer-implemented tasks are described as being performed by a particular entity, other embodiments are possible in which these tasks are performed by a different entity.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

While the above detailed description has shown, described, and pointed out novel features as applied to various embodiments, it will be understood that various omissions, substitutions, and changes in the form and details of the devices or algorithms illustrated can be made without departing from the spirit of the disclosure. As will be recognized, the processes described herein can be embodied within a form that does not provide all of the features and benefits set forth herein, as some features can be used or practiced separately from others. The scope of protection is defined by the appended claims rather than by the foregoing description. All changes which come within the meaning and range of equivalency of the claims are to be embraced within their scope.

What is claimed is:

1. A composition for treatment of pain and inflammation in humans, the composition comprising:
4.0-10.0% Diclofenac Sodium by weight;
2.0-6.0% Gabapentin by weight;
1.0-2.0% Baclofen by weight; and
2.0-5.0% Cyclobenzaprine by weight.

2. The composition of claim 1, wherein the composition is in the form of a lotion.

3. The composition of claim 1, wherein the composition is in the form of a cream.

4. The composition of claim 1, comprising at least one inactive ingredient.

5. A composition for treatment of pain and inflammation in humans, the composition comprising:
0.5-1.0% Bupivacaine by weight;
2.0-6.0% Gabapentin by weight;
1.0-2.0% Baclofen by weight;
1.0-2.0% Cyclobenzaprine by weight; and
3.0-5.0% Diclofenac Sodium by weight.

6. The composition of claim 5, wherein the composition is in the form of a lotion.

7. The composition of claim 5, wherein the composition is in the form of a cream.

8. The composition of claim 5, comprising at least one inactive ingredient.

9. A composition for treatment of pain and inflammation in humans, the composition comprising:
5.0-10.0% Ketoprofen by weight;
1.0-2.0% Baclofen by weight;
1.0-5.0% Gabapentin by weight; and
4.0-6.0% Lidocaine by weight.

10. The composition of claim 9, wherein the composition is in the form of a lotion.

11. The composition of claim 9, wherein the composition is in the form of a cream.

12. The composition of claim 9, comprising at least one inactive ingredient.

13. A composition for treatment of pain and inflammation in humans, the composition comprising:
5.0-10.0% Flurbiprofen by weight;
1.0-5.0% Cyclobenzaprine by weight;
2.0-6.0% Gabapentin by weight;
1.0-2.0% Lidocaine by weight; and
1.0-2.0% Prilocaine by weight.

14. The composition of claim 13, wherein the composition is in the form of a lotion.

15. The composition of claim 13, wherein the composition is in the form of a cream.

16. The composition of claim 13, comprising at least one inactive ingredient.

17. A composition for treatment of pain and inflammation in humans, the composition comprising:
2.0-6.0% Gabapentin by weight;
1.0-2.5% Lidocaine by weight; and
0.1-0.2% Clonidine by weight.

18. The composition of claim 17, wherein the composition is in the form of a lotion.

19. The composition of claim 17, wherein the composition is in the form of a cream.

20. The composition of claim 17, comprising at least one inactive ingredient.
